(19) United States
(12) Patent Application Publication
(10) Pub. No.: US 2014/0261420 A1
(43) Pub. Date: Sep. 18, 2014

(54) PRESSURE CONTROL FOR RESPIRATORY HUMIDIFICATION APPARATUS

(71) Applicant: TELEFLEX MEDICAL INCORPORATED, Research Triangle Park, NC (US)

(72) Inventors: Daniel Patrick DWYER, Raleigh, NC (US); Jason FOUSHEE, Durham, NC (US); Marc Alan BURK, Menifee, CA (US)

(21) Appl. No.: 14/204,830

(22) Filed: Mar. 11, 2014

Related U.S. Application Data
(60) Provisional application No. 61/779,463, filed on Mar. 13, 2013.

(51) Int. Cl. A61M 16/16

(52) U.S. Cl. CPC A61M 16/164
USPC 128/204.14

(57) ABSTRACT

A humidifier assembly includes a water supply reservoir; and a vessel defining a water holding cavity configured to hold water at a water level, and having an inlet adjacent to the bottom thereof, a respiratory gas inlet, a respiratory gas outlet, a gas pressure equalization port, a water level control port, and a water level control pipe communicating with said water level control port and configured to extend below the water level in the cavity. Tubing directs water from the water supply container to said humidifier vessel. Tubing also extends between the water supply container and gas pressure equalization port and water level control port. A first one-way valve is disposed along a pressure equalization flow path between the reservoir and cavity, which path includes the gas pressure equalization port, with third tubing extending therefrom to an opposite end disposed under the water level in the water holding cavity.

### CROSS REFERENCE TO RELATED APPLICATION

This application claims the benefit of U.S. Provisional Application No. 61/779,463, filed on Mar. 13, 2013, the disclosure of which is hereby incorporated by reference in its entirety.

### FIELD OF THE INVENTION

The present invention relates to respiratory apparatus of medical applications, specifically respiratory humidification systems and pressure regulation and control for such systems.

### BACKGROUND

Humidifying apparatus such as disclosed in U.S. Pat. Nos. 4,110,419, 4,172,105, 4,366,105, 4,500,480, 4,674,494, 4,765,327 and 6,050,552 describe different variations of cartridge-type humidification systems and apparatus incorporating a heated humidifier module connected via tubing to a water supply reservoir. In these systems, the humidification chamber is installed within a heater receptacle. As water is evaporated in the heated humidification chamber replacement water is fed to the cavity from the adjacent water supply reservoir. The descriptions of the aforesaid patents are incorporated herein by reference.

In U.S. Pat. Nos. 4,500,480 and 6,050,552, a water level sensing tube positioned in the humidification chamber communicates with the air space above the water level in the water supply reservoir via tubing with a one-way valve. Water is fed to the humidification chamber from the bottom of the reservoir via tubing, also provided with a one-way valve. In FIG. 1, such a prior art humidifier assembly is shown. The assembly includes a hollow humidifier vessel 10 with a water holding cavity 11, the vessel having a lid or cap 16 for enclosing the cavity. The cap is provided with a respiratory gas outlet 12, respiratory gas inlet 14 and a water level control port 18 communicating exteriorly of the humidifier vessel through a tubing adapter formed thereon. The respiratory gas inlet 14 is for receiving gas through suitable gas delivery tubing (not shown) from a respirator, and the outlet 12 secured to respiratory tubing (not shown) for directing humidified gas from the humidifier vessel to a patient, typically through a face mask, nose mask, tracheotomy mask or the like. The humidifier vessel 10 is also provided with a water level control pipe 20, the upper end of which is attached to an adapter extending from or communicating with water level control port 18. At the bottom or lower end of the humidifier vessel is a nipple or adapter 22 for directing water to the cavity.

The humidifier assembly also includes a water supply container and reservoir 30 having a port 32 adjacent to the upper end or top of the reservoir and communicating with the interior space of the reservoir, above the water level. Adjacent to the bottom end of the reservoir 30 is a port 34. Water from the reservoir is supplied via tubing 28, one end which is attached to port 34 on the reservoir 30 and the other end to water inlet nipple 22 of the humidifier vessel. The water supply tubing 28 is provided with a check valve or one-way valve 26 which allows water to be directed from the reservoir 30 to the humidifying vessel 10. Tubing 25 adjacent to the upper portion of the assembly is secured to port 32 on the water reservoir 30 and to water level control port 18 on the cap 16 of the humidifier vessel 10. Tubing 25 is also provided with a check valve or one-way valve 23 which allows fluid to pass from the humidifier vessel to the water reservoir. Further description and operation of the apparatus shown in FIG. 1 is described, for example, in U.S. Pat. No. 4,500,480.

During continuous gas flow, for example, where the humidification apparatus is used with a CPAP system or is connected to a respirator or ventilator which directs respiratory gas into the humidification chamber under pressure, the humidification chamber can be is exposed to pressures of 150 cm H₂O or more during continuous gas flow, or pressures in excess of 120 cm H₂O during intermittent gas flow. However, sudden pressure reduction in the humidification chamber can result in rapid transfer of water from the reservoir and flooding of the chamber and even into the respiratory gas tubing to a patient.

U.S. Pat. No. 7,228,859, the disclosure of which is incorporated herein by reference in its entirety, addresses and solves this problem and is described in FIGS. 2 and 3. Observing FIG. 2, a pressure equalizing assembly or apparatus includes tubing 31 which communicates the interior cavity of humidifier vessel 10 with the interior cavity of water supply reservoir 30. One end of tubing 31 is secured to gas pressure vent or pressure equalization port 29 on humidifier vessel cap 16. The other end of tubing 31 is secured to a flow splitting device shown as a tee adapter along tubing 25. Tubing 31 is also provided with a check valve or one-way valve 33 whereby fluid may pass only from reservoir 30 to the humidifier vessel 10. It will also be observed that the tubing 31 communicates with port 32 above water level 19 in the water reservoir, and above water level 17 in humidifier vessel 10. Thus, the pressure equalizing assembly communicates with air space 27 in reservoir 30 and air space 24 in humidifier vessel 10. Although both one-way valves 23 and 33 are shown in FIG. 2 as located along tubing 25 and 31, respectively, either or both of these one-way valves may be located in the tubing adapters or in a tee adapter.

In operation of the humidifier assembly shown in FIGS. 1 and 2, water from reservoir 30 is supplied via tubing 28 from the bottom of the reservoir at outlet port 34 to the bottom of water holding cavity 11 in humidifier vessel 10 through nipple 22 at the bottom of the vessel. One-way valve 26 prevents water from returning to the reservoir 30 from the humidifier vessel 10. As relatively dry respiratory gas is introduced into the humidifier vessel, it contacts heated water in the water holding cavity 11 and is humidified. The humidified gas is then directed via respiratory gas outlet 12 to a patient. As the water within the humidifier vessel evaporates, the water level is lowered, and the water level is sensed by water level control pipe 20, the bottom end 21 of which contacts and is submerged in the water. One end of tubing 25, communicates with the reservoir space 27 above the water level 19, and the other end with the water level control pipe 20 via water level control port 18. Thus, the water level control pipe maintains a substantially constant liquid level in the humidification chamber, whereby the water level in pipe 20 is the same as the water level in reservoir 30.

During operation of the apparatus which is connected to a ventilator, pressure in the humidifier vessel increases relative to the reservoir 30 as previously discussed. This relatively high pressure in the humidifier vessel is transferred to the reservoir via tubing 25 and one-way valve 23 whereby the water level in pipe 20 equalizes the pressure between the two containers. When pressure in the humidifying vessel is suddenly changed however, for example, when the pressurized vessel is disconnected from a respirator, a substantial pressure gradient can be created between the humidification chamber and the reservoir. In the prior art apparatus illustrated in FIG. 1, when such a pressure gradient is suddenly created, water is rapidly transferred through tubing 28 via one-way valve 26 until equilibrium is reached between the humidification chamber and the reservoir. Such equilibrium can result in excessive amounts of water from the reservoir flooding the humidification chamber which may also undesirably cause delivery of such water to the patient from the humidifying vessel via respiratory outlet 12.

Undesirable flooding of the humidifier vessel is prevented by the apparatus illustrated in FIG. 2. A substantial reduction in pressure in the humidifier vessel as described above will be equalized via tubing 31 and one-way valve 33 as pressure is equalized in space 24 in the humidifier vessel and space 27 in the reservoir 30, without substantial movement of water from the reservoir to the humidifier vessel in response to such pressure reduction. Communication of space 27 in reservoir 30 and space 24 in humidifier vessel 10 via tubing 31 allows transfer of gas from the reservoir to the humidification chamber above the water level also avoiding creation of aerosols or other particulate water from being delivered to a patient in response to any substantial pressure reduction within vessel 10. Such a configuration allows the reservoir to vent gas back to the humidification chamber without transfer of substantial fluid through the tubing 28 and one-way valve 26 when the pressure in the humidification chamber is substantially reduced.

FIG. 3 illustrates another embodiment in which the pressure equalizing valves are located inside the humidifier vessel. In the embodiment shown, a first check valve or one-way valve 23 is located in the water level control pipe 20 and a second check valve or one-way valve 33 is located in a vent port pipe or gas pressure equalization port 40. These one-way valves 23 and 33 allow gas to flow only through the valve in the direction of the arrows illustrated. It will be observed that both of these valves operatively communicate with water level control port 18 which extends from cover or lid 16 of the humidifier vessel 10. Interior piping, tubing or adapters are formed on the underside of lid 16 to provide communication of the one-way valves and inlet vent port pipe 40 and water level control pipe 20 with the water level control port. An advantage of the embodiment shown in FIG. 3 is that only a single tube 25 is used for equalizing pressures in the water supply container 30 and humidifier vessel 10. The embodiment of FIG. 3 provides pressure equalization between the air space 27 in reservoir 30 with the air space 24 in humidifier vessel 10 via the single tubing 25. Thus, transfer of gas from the reservoir to the humidification chamber above the water level via vent port pipe 40 and one-way valve 33 is provided through the same tubing as used for the water level control via water level control pipe 20 as previously described.

In both of the embodiments of FIGS. 2 and 3, during continuous gas flow through the humidifier vessel, the pressure in the reservoir space 27 can at times be lower than that in the humidifier air space 24, thereby allowing a water column to be maintained in the water level control pipe 20. However one problem which can occur in the apparatus of FIGS. 2 and 3 is that the one way valve 33, if faulty or poorly designed, can leak and allow gas to transfer, at times slowly, from the humidifier air space 24 to the reservoir space 27, thereby canceling the existence of the water column in water level control pipe 20 and having negative consequences for the set patient ventilation values while operating the apparatus, such as loss of delivery tidal volume.

Therefore, it is desired to provide a new pressure equalizing apparatus installed in a humidifier assembly to address this problem of a leaky or failed one-way valve 33 in the apparatus as disclosed in FIGS. 2 and 3, so as to maintain a consistent and optimal water level in the humidifier vessel 10.

### SUMMARY OF THE INVENTION

In a first embodiment of the present invention, a humidifier assembly includes a water supply container enclosing a water containing reservoir; a humidifier vessel defining a water holding cavity configured to hold water at a water level therein, and having an inlet adjacent to the bottom thereof, a respiratory gas inlet, a respiratory gas outlet, a gas pressure equalization port, a water level control port, and a water level control pipe communicating with said water level control port and configured to extend below the water level in the water holding cavity. First piping comprises tubing for directing water from said water supply container to said humidifier vessel. Second piping comprises tubing extending between said water supply container and each of (i) said gas pressure equalization port and (ii) said water level control port. A first one-way valve is disposed to provide flow communication along a pressure equalization flow path between a gas space in the water containing reservoir and the water holding cavity in the humidifier vessel, the gas pressure equalization port being disposed along said flow path. And third piping comprises tubing extending at a first end from the gas pressure equalization port to an opposite end disposed under the water level in the water holding cavity.

In another embodiment, the first one-way valve is disposed in the tubing of the second piping between the water supply container and the gas pressure equalization port for directing gas flow from said water supply container to said gas pressure equalization port.

In another embodiment, a second one-way valve is disposed in the tubing of the second piping between the water supply container and water level control port for directing flow from said water level control pipe to said water supply container.

In another embodiment, the first one-way valve is disposed in the tubing of the third piping and communicating with the gas pressure equalization port for directing gas flow from said water supply container, through said gas pressure equalization port, and into the water holding cavity, said first one-way valve being disposed under the water level in the water holding cavity.

In another embodiment, a third one-way valve cooperates with said first piping for directing flow of water from said water supply container to said humidifier vessel.

In another embodiment of the present invention, a humidifier assembly includes a water supply container enclosing a water containing reservoir; a humidifier vessel defining a cavity configured to hold water at a water level, and having an inlet adjacent to the bottom thereof, a respiratory gas inlet, a respiratory gas outlet, a water level control port, and a water level control pipe communicating with said water level control port and configured to extend below the water level in the water holding cavity, the water level control port further defining a gas pressure equalization port communicating between the water level control pipe and the cavity. First piping comprises tubing for directing water from said water supply container to said humidifier vessel. Second piping comprises tubing extending between said water supply container and water level control port. A first one-way valve is disposed to provide flow communication along a pressure equalization flow path between a gas space in the water containing reservoir and the water holding cavity in the humidifier vessel, the gas pressure equalization port being disposed along said flow path. Third piping comprises tubing extending at a first end from the gas pressure equalization port to an opposite end disposed under the water level in the water holding cavity.

In another embodiment, a second one-way valve is disposed in the water level control pipe for directing flow from said water level control pipe to said water supply container.

In another embodiment, the first one-way valve is disposed in the tubing of the third piping and communicates with the gas pressure equalization port for directing gas flow from said water supply container, through said second piping and said gas pressure equalization port, and into the water holding cavity.

In another embodiment, said first one-way valve is disposed under the water level in the water holding cavity.

In another embodiment, said first one-way valve is disposed in the gas pressure equalization port.

In another embodiment, a third one-way valve cooperates with said first piping for directing flow of water from said water supply container to said humidifier vessel.

In another embodiment of the present invention, a humidifier vessel defining a water holding cavity configured to hold water at a water level therein, comprises an inlet adjacent to a bottom end of said water holding cavity, a respiratory gas inlet and a respiratory gas outlet each communicating with said water holding cavity adjacent to a top of said water holding cavity, a first port and a generally vertical pipe having an open upper end communicating with said first port and an open lower end for contacting water in said water holding cavity, and a second port communicating with said water holding cavity adjacent to the top of said water holding cavity. A water supply container encloses a water containing reservoir. First tubing has one end communicating with said water holding cavity via said inlet and another end communicating with said water containing reservoir adjacent to the bottom thereof. A first one-way valve cooperates with said first tubing for directing water from said water supply container to said humidifier vessel. Second tubing has one end communicating with said first port and another end communicating with said water containing reservoir above water level therein. A second one-way valve cooperates with said second tubing for directing fluid from said pipe to said water containing reservoir. Third tubing has a first end communicating with said second port and a second end communicating with said water containing reservoir above water level therein. And fourth tubing extends at a first end from said second port to an opposite end disposed under the water level in the water holding cavity.

In another embodiment, a third one-way valve cooperates with said third tubing for directing gas from said water containing reservoir to said water holding cavity.

In another embodiment, a third one-way valve cooperates with said fourth tubing for directing gas from said water containing reservoir to said water holding cavity, the third one-way valve being disposed under the water level in the water holding cavity.

In another embodiment, said second end of said third tubing communicates with said second tubing between said second one-way valve and said water containing chamber.

### BRIEF DESCRIPTION OF THE DRAWINGS

FIG. 1 illustrates a prior art humidifier assembly including a water supply reservoir, a humidifier vessel including a humidification chamber, and tubing and one-way valves communicating the humidifier vessel and water supply reservoir;

FIG. 2 illustrates another prior art humidifier assembly including one embodiment of a pressure equalizing apparatus installed in a humidifier assembly of FIG. 1;

FIG. 3 illustrates another prior art humidifier assembly including another embodiment of a pressure equalizing apparatus installed in a humidifier assembly of FIG. 1, with the valving installed within the humidifier vessel;

FIGS. 4 and 5 illustrate embodiments of the present invention having new pressure equalizing apparatus installed in a humidifier assembly such as in FIG. 2; and

FIGS. 6 and 7 illustrate embodiments of the present invention having new pressure equalizing apparatus installed in a humidifier assembly such as in FIG. 3.

### DETAILED DESCRIPTION

Turning now to the drawings, in which like reference characters indicate corresponding elements throughout the several views, FIGS. 4 and 5 illustrate embodiments of the present invention having a new pressure equalizing apparatus installed in a humidifier assembly such as in FIG. 2. The humidifier assemblies shown in FIGS. 4 and 5 include the basic apparatus of the humidifier assembly shown in FIG. 2, where a pressure equalizing assembly or apparatus includes tubing 31 which communicates the interior cavity of humidifier vessel 10 with the interior cavity of a water supply reservoir 30. One end of tubing 31 is secured to a gas pressure vent or pressure equalization port 29 on a humidifier vessel cap 16. The other end of tubing 31 is secured to a flow splitting device such as a tee adapter along tubing 25. Thus, the piping extending between a port 32 on the water reservoir 30 and the humidifier vessel 10 branches into two lengths of tubing, including a tubing 31 which is also provided with a check valve or one-way valve 33 whereby fluid may pass only from reservoir 30 to the humidifier vessel 10. Tubing 31 communicates with port 32 above the water level 19 in the water reservoir 30, and above the water level 17 in the humidifier vessel 10. In operation, water from reservoir 30 is supplied via tubing 28 from the bottom of the reservoir 30 at outlet port 34 to the bottom of water holding cavity 11 in humidifier vessel 10 through nipple 22 at the bottom of the vessel. One-way valve 26 prevents water from returning to the reservoir 30 from the humidifier vessel 10. As relatively dry respiratory gas is introduced into the humidifier vessel, it contacts heated water in the water holding cavity 11 and is humidified. The humidified gas is then directed via respiratory gas outlet 12 to a patient. As the water within the humidifier vessel 10 evaporates, the water level 17 is lowered, and is sensed by water level control pipe 20, the bottom end 21 of which contacts and is submerged below the water level 17. One end of tubing 25, communicates with the reservoir space 27 above the water level 19 in reservoir 30, and the other end with the water level control pipe 20 via water level control port 18. Thus, the water level control pipe maintains a substantially constant liquid level in the humidification chamber.

The key difference between the apparatus of FIGS. 4 and 5, and that of FIG. 2, is that tubing or piping 31A extends inside the humidifier vessel 10 below and from the pressure equalization port 29 down to an opening or opposite end 31B below the water level 17 in the humidifier vessel 10. This prevents any leak in the one-way valve 33 from transferring pressure from the humidifier vessel space 24 back to the reservoir space 27, when a pressure differential exists therebetween. The positioning of the piping 31A with opening 31B effectively operates as a “water seal” to prevent any leakage in the one way valve 33 from compromising the intended operation of the humidifier vessel 10, which requires that the water level 17 be maintained at a consistent level.

Thus, in a first embodiment of the present invention, a humidifier assembly comprises a water supply container enclosing a water containing reservoir 30, the humidifier vessel 10 defining a water holding cavity 11 configured to hold water at a water level 17 therein, and having an inlet 22 adjacent to the bottom thereof, a respiratory gas inlet 14, a respiratory gas outlet 12, a gas pressure equalization port 29, a water level control port 18, and a water level control pipe 20 communicating with said water level control port 29 and configured to extend below the water level 17 in the water holding cavity 11. First piping includes tubing 28 for directing water from said water supply container 30 to said humidifier vessel 10. Second piping includes tubing 31 extending between the water supply container 30 and the gas pressure equalization port 29, and tubing 25 extending between said water supply container 30 and the water level control port 18. A one-way valve 33 is disposed to provide flow communication along a pressure equalization flow path between gas space 27 in the water containing reservoir 30 and the water holding cavity 11 in the humidifier vessel, the gas pressure equalization port 29 being disposed along said flow path. Third piping includes tubing 31A extending at a first end from the gas pressure equalization port 29 to an opposite end 31B disposed under the water level 17 in the water holding cavity 11. This effectively seals and prevents gas flow from space 24 inside the humidifier vessel 10 in the direction opposite to the one-way valve 33 to the reservoir space 27.

Thus, the present invention still prevents undesirable flooding of the humidifier vessel 10 via tubing 31 and one-way valve 33 as pressure is equalized in space 24 in the humidifier vessel 10 and space 27 in the reservoir 30 in the event of a sudden negative pressure gradient between space 24 and space 27, without substantial movement of water from the reservoir 30 to the humidifier vessel 10 in response to such pressure reduction. Communication of space 27 in reservoir 30 and space 24 in humidifier vessel 10 via tubing 31 allows transfer of gas along a pressure equalization flow path from the reservoir to the humidification chamber above the water level also avoiding creation of aerosols or other particulate water from being delivered to a patient in response to any substantial pressure reduction within vessel 10. Such a configuration allows the reservoir to vent gas back to the humidification chamber without transfer of substantial fluid through the tubing 28 and one-way valve 26 when the pressure in the humidification chamber is substantially reduced. Yet the present invention also prevents the water level 17 from being compromised and altered by a faulty or leaky one-way valve 33, since tubing 31 effectively extends all the way under the water level 17 inside of humidifier vessel 10, effectively sealing such valve 33.

As shown in FIG. 4, one embodiment of the present invention includes one-way valve 33 disposed in the tubing 31 between the water supply container 30 and the gas pressure equalization port 29 for directing gas flow from said water supply container 30 to said gas pressure equalization port 29. As shown in FIG. 5, another embodiment of the present invention includes one-way valve 31C disposed in the tubing 31A communicating with the gas pressure equalization port 29 for directing gas flow from said water supply container 30, through said gas pressure equalization port 29, and into the water holding cavity 11, the one-way valve 31C being disposed under the water level 17 in the water holding cavity 11. In such an embodiment, there is no one-way valve 33 locating in the tubing 31 outside of the humidifier vessel 10; instead one-way valve 31C oriented the same direction as valve 33 is integrated with tubing 31A, thereby providing a simpler and potentially more cost-effective device.

FIGS. 6 and 7 illustrate embodiments of the present invention having a new pressure equalizing apparatus installed in a humidifier assembly such as in FIG. 3. The humidifier assemblies shown in FIGS. 6 and 7 include the basic apparatus of the humidifier assembly shown in FIG. 3, where a first check valve or one-way valve 23 is located in the water level control pipe 20. In the embodiment of FIG. 6, a second check valve or one-way valve 33 is located between the gas pressure equalization port 40 communicating between the water level control pipe 20 and the cavity 11 of the humidifier vessel 10. These one-way valves 23 and 33 allow gas to flow only through the valve in the direction of the arrows illustrated. An advantage of the embodiment shown in FIGS. 6 and 7 is that only a single tube 25 is used for equalizing pressures between the water supply container 30 and humidifier vessel 10. Thus, transfer of gas from the reservoir 30 to the humidification chamber 11 above the water level 17 via gas pressure equalization port 40 and one-way valve 33 is provided through the same tubing as used for the water level control via water level control pipe 20 as previously described.

The key difference between the apparatus of FIGS. 6 and 7, and that of FIG. 3, is that tubing or piping 33A extends inside the humidifier vessel 10 below and from the gas pressure equalization port 40 down to an opening or opposite end 33B below the water level 17 in the humidifier vessel 10. This prevents any leak in the one-way valve 33 from transferring pressure from the humidifier vessel space 24 back to the reservoir space 27, when a pressure differential exists there between. The positioning of the piping 33A with lower opening 33B under water level 17 effectively operates as a “water seal” to prevent any leakage in the one way valve 33 from compromising the intended operation of the humidifier vessel 10, which requires that the water level 17 be maintained at a consistent level.

Thus, in another embodiment of the present invention, a humidifier assembly comprises water supply container enclosing a water containing reservoir 30, humidifier vessel 10 defining cavity 11 configured to hold water at a water level 17, and having an inlet 22 adjacent to the bottom thereof, a respiratory gas inlet 14, a respiratory gas outlet 12, a water level control port 18, and a water level control pipe 20 communicating with said water level control port 18 and configured to extend below the water level 17 in the water holding cavity 11, the vessel 10 further defining a gas pressure equalization port 40 communicating between the water level control pipe 20 and the cavity 11. First piping includes tubing 28 for directing water from the water supply container 30 to the humidifier vessel 22. Second piping includes tubing 25 extending between the water supply container 30 and water level control port 18. A one-way valve 33 is disposed to provide flow communication along a pressure equalization flow path between gas space 27 in the water containing reservoir 30 and the water holding cavity 11 in the humidifier vessel 10, the gas pressure equalization port 40 being disposed along said flow path. Third piping includes tubing 33A extending at a first end from the gas pressure equalization port 40 to an opposite end 33B disposed under the water level 17 in the water holding cavity 11. As shown in FIGS. 6 and 7, embodiments of the present invention includes one-way valve 23 disposed in the water level control pipe 20 for directing flow from said water level control pipe 20 to the water supply container 30. As shown in FIG. 6, another embodiment of the present invention includes one-way valve 33 disposed in the tubing 33A communicating with the gas pressure equalization port 40 for directing gas flow from the water supply container 30, through second piping tube 25 and gas pressure equalization port 40, into the water holding cavity 11. In one embodiment, as shown in FIG. 7, a one-way valve 33C is disposed under the water level 17 in the water holding cavity 11 at the end of tubing 33A above its end 33B. In another embodiment, one-way valve 33 may be disposed in the gas pressure equalization port 40 itself.

The many features and advantages of the present invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A humidifier assembly comprising:
a water supply container enclosing a water containing reservoir;
a humidifier vessel defining a water holding cavity configured to hold water at a water level therein, and having an inlet adjacent to the bottom thereof, a respiratory gas inlet, a respiratory gas outlet, a gas pressure equalization port, a water level control port, and a water level control pipe communicating with said water level control port and configured to extend below the water level in the water holding cavity;
first piping comprising tubing for directing water from said water supply container to said humidifier vessel;
second piping comprising tubing extending between said water supply container and each of (i) said gas pressure equalization port and (ii) said water level control port;
a first one-way valve disposed to provide flow communication along a pressure equalization flow path between a gas space in the water containing reservoir and the water holding cavity in the humidifier vessel, the gas pressure equalization port being disposed along said flow path; and
third piping comprising tubing extending at a first end from the gas pressure equalization port to an opposite end disposed under the water level in the water holding cavity.

2. The humidifier assembly of claim 1, further comprising:
a second one-way valve disposed in the tubing of the second piping between the water supply container and water level control port for directing flow from said water level control pipe to said water supply container.

3. The humidifier assembly of claim 1,
wherein the first one-way valve is disposed in the tubing of the third piping and communicating with the gas pressure equalization port for directing gas flow from said water supply container, through said gas pressure equalization port, and into the water holding cavity, said first one-way valve being disposed under the water level in the water holding cavity.

4. A humidifier of assembly of claim 1, further comprising a third one-way valve cooperating with said first piping for directing flow of water from said water supply container to said humidifier vessel.

5. The humidifier assembly of claim 1,
wherein the first one-way valve is disposed in the tubing of the second piping between the water supply container and the gas pressure equalization port for directing gas flow from said water supply container to said gas pressure equalization port.

6. The humidifier assembly of claim 5, further comprising:
a second one-way valve disposed in the tubing of the second piping between the water supply container and water level control port for directing flow from said water level control pipe to said water supply container.

7. The humidifier assembly of claim 6,
wherein the first one-way valve is disposed in the tubing of the third piping and communicating with the gas pressure equalization port for directing gas flow from said water supply container, through said gas pressure equalization port, and into the water holding cavity, said first one-way valve being disposed under the water level in the water holding cavity.

8. A humidifier of assembly of claim 7, further comprising a third one-way valve cooperating with said first piping for directing flow of water from said water supply container to said humidifier vessel.

9. A humidifier assembly comprising:
a water supply container enclosing a water containing reservoir;
a humidifier vessel defining a cavity configured to hold water at a water level, and having an inlet adjacent to the bottom thereof, a respiratory gas inlet, a respiratory gas outlet, a water level control port, and a water level control pipe communicating with said water level control port and configured to extend below the water level in the water holding cavity, the vessel further defining a gas pressure equalization port communicating between the water level control pipe and the cavity;
first piping comprising tubing for directing water from said water supply container to said humidifier vessel;
second piping comprising tubing extending between said water supply container and water level control port;
a first one-way valve disposed to provide flow communication along a pressure equalization flow path between a gas space in the water containing reservoir and the water holding cavity in the humidifier vessel, the gas pressure equalization port being disposed along said flow path; and
third piping comprising tubing extending at a first end from the gas pressure equalization port to an opposite end disposed under the water level in the water holding cavity.

10. The humidifier assembly of claim 9,
wherein the first one-way valve is disposed in the tubing of the third piping and communicating with the gas pressure equalization port for directing gas flow from said water supply container, through said second piping and said gas pressure equalization port, and into the water holding cavity.

11. The humidifier assembly of claim 10, said first one-way valve being disposed under the water level in the water holding cavity.

12. The humidifier assembly of claim 10, said first one-way valve being disposed in the gas pressure equalization port.

13. A humidifier of assembly of claim 9, further comprising a third one-way valve cooperating with said first piping for directing flow of water from said water supply container to said humidifier vessel.

14. The humidifier assembly of claim 9, further comprising:
a second one-way valve disposed in the water level control pipe for directing flow from said water level control pipe to said water supply container.

15. The humidifier assembly of claim 14,
wherein the first one-way valve is disposed in the tubing of the third piping and communicating with the gas pressure equalization port for directing gas flow from said water supply container, through said second piping and said gas pressure equalization port, and into the water holding cavity.

16. The humidifier assembly of claim 15, said first one-way valve being disposed under the water level in the water holding cavity.

17. The humidifier assembly of claim 15, said first one-way valve being disposed in the gas pressure equalization port.

18. A humidifier assembly comprising:
a humidifier vessel defining a water holding cavity configured to hold water at a water level therein, the vessel comprising an inlet adjacent to a bottom end of said water holding cavity, a respiratory gas inlet and a respiratory gas outlet each communicating with said water holding cavity adjacent to a top of said water holding cavity, a first port and a generally vertical pipe having an open upper end communicating with said first port and an open lower end for contacting water in said water holding cavity, and a second port communicating with said water holding cavity adjacent to the top of said water holding cavity;
a water supply container enclosing a water containing reservoir;
first tubing having one end communicating with said water holding cavity via said inlet and another end communicating with said water containing reservoir adjacent to the bottom thereof, and a first one-way valve cooperating with said first tubing for directing water from said water supply container to said humidifier vessel;
second tubing having one end communicating with said first port and another end communicating with said water containing reservoir above water level therein, and a second one-way valve cooperating with said second tubing for directing fluid from said pipe to said water containing reservoir;
third tubing having a first end communicating with said second port and a second end communicating with said water containing reservoir above water level therein; and
fourth tubing extending at a first end from said second port to an opposite end disposed under the water level in the water holding cavity.

19. A humidifier assembly of claim 18, further comprising a third one-way valve cooperating with said third tubing for directing gas from said water containing reservoir to said water holding cavity.

20. A humidifier assembly of claim 18, further comprising a third one-way valve cooperating with said fourth tubing for directing gas from said water containing reservoir to said water holding cavity, the third one-way valve being disposed under the water level in the water holding cavity.

21. A humidifier assembly of claim 18, wherein said second end of said third tubing communicates with said second tubing between said second one-way valve and said water containing chamber.
